(19) United States
(12) Patent Application Publication
(10) Pub. No.: US 2011/0117523 A1
(43) Pub. Date: May 19, 2011

(54) ABRASIVE AGENTS

(75) Inventors: Richard James Cook, London (GB); Timothy F. Watson, London (GB); Ian D. Thompson, London (GB)

(73) Assignee: Kings College London, London (GB)

(21) Appl. No.: 12/991,940

(22) PCT Filed: May 21, 2009
(86) PCT No.: PCT/GB2009/001273

§ 371 Date: Jan. 2, 2011

(30) Foreign Application Priority Data
May 21, 2008 (GB) 0809253.8

(51) Int. Cl. A61K 8/81; A61Q 11/00; A61C 17/00; A61C 19/04

(52) U.S. Cl. 433/216; 424/43

(57) ABSTRACT

The present invention relates to the use of a polyalkenoate acid, such as polyacrylic acid or polymaleic acid, in the manufacture of an air abrasive agent for use in conditioning dental tissue, methods of using such air abrasive agents, dental compositions comprising a polyalkenoate acid, air abrasive agents comprising a polyalkenoate acid and at least one of a bioactive glass, a glycine salt, baking soda and alumina, methods of treatment and/or prophylaxis using an air abrasive agent comprising a polyalkenoate acid.

The present invention relates to abrasive agents suitable for use in conditioning dental tissue.

Dental pain is a common problem affecting 17% of the population at any one time and increases in frequency with age (Litkowski L J., Hack G D., Sheaffer H B., Greenspan D C., 1997, Occlusion of dentine tubules by 45S5 Bioglass®, Bioceramics 10 Procs 10^{th} Int., Symposium on ceramics in Medicine pp 411-414, Ed. Sedel L & Rey C. Elsevier Science Ltd.). The principle causes of this dental discomfort arise from both abrasion and acid erosion of external root dentine, which has either been revealed after gingival recession (due to periodontal disease or as a feature of maintaining teeth further into old age) or has developed due to dental caries i.e. the acid dissolution of dental tissues resulting from bacterial plaque metabolic activity.

One of the currently accepted causes of dental pain is the hydrodynamic theory (Litkowski, et al 1997) whereby movement of fluid through the dentine tubule tracts excites either the odontoblasts, whose processes (once) occupied the tubules, or adjacent nerve fibres directly. In the normal healthy state, these tubules are closed off by the overlying enamel crown or cementum of the root. When dentine is exposed the tubules are lain open, allowing large fluid movements and consequential dental pain.

Dental pain resulting from cavity formation is usually treated by removal of the carious dental tissue and restoration by filling. Dental fillings may be fabricated out of a variety of materials, including dental amalgam, glass ionomer cement and resin composites. Amalgam is a mixture of mercury with at least one other metal and it has been used as a dental filling material for over 150 years. Amalgam is a low cost, high strength material that produces highly durable fillings, however, it is not tooth-coloured and concerns exist due to the fact that it contains a toxic metal (mercury). Glass ionomer cement is based on the reaction of alkaline glass and polyacrylic acid. These white-coloured materials were introduced in the 1970s for use on anterior teeth. Glass ionomer cements are self-adhesive, forming chemical bonds with hard dental tissues. Resin composites are a group of materials used in dentistry that typically consist of a resin-based matrix and an inorganic filler, such as silica, ceramic or quartz. The filler gives the composite wear resistance and translucency, producing a tooth-coloured filling. The resin-based matrix gives the composite structure and may adhere to conditioned enamel; however, resin composites are commonly used with a separate adhesive to ensure a strong bond between filling and tooth. Polymerization of the resin-based matrix begins when electromagnetic radiation (light/heat etc.) is applied, causing the resin-based matrix to mix with the conditioned enamel and/or adhesive.

Preparing a tooth prior to filling using one of the above materials commonly requires the carious dentine to be removed with a drill, which leaves a smear layer of disaggregated tissue (loose dentine chippings, loose enamel chippings, bacteria etc.) impacted into the surface of the tooth. If a glass ionomer cement or resin composite is going to be used as the filling material, then the smear layer must be removed by etching of the tooth surface with an acid, such as phosphoric acid, prior to the filling material being applied. If a composite material is going to be used, an adhesive will commonly be applied after the acid, or a ‘self-etching’ system may be used which comprises the acid in combination with an adhesive. Current adhesive materials contain water which can be difficult to remove. Residual water can lead to hydrolytic breakdown of the adhesive, which can weaken the bond between the filling and the tooth and may expose dentine resulting in dental pain. Therefore there exists a need for improved methods and materials for preparing (conditioning) dental tissue (teeth) during the treatment of dental disorders.

The avoidance of rotary cutting instruments in the clinical dental context has long been identified as a beneficial development, as the sounds, vibrations and mechanical pressures associated with the use of dental drills are frequently identified as sources of anxiety and apprehension among the general population. Air abrasion as a means of cutting or conditioning tooth substrate surfaces by harnessing the transferred kinetic energy of alumina particles accelerated in a controlled compressed gas stream has been known since the 1950s. (Note that the use of other gases as a propellant (e.g. CO₂ or N₂) is included in the definition of “air abrasion”.) The abrasive stream cuts (abrades) through the target substrate by repeated localised impacts serially removing material from the point of aim. Some air abrasive systems also employ aqueous irrigation systems, co-localised to the air abrasive gas stream to improve cutting and debris washout.

However, the harder and more brittle the target (e.g. sound enamel, dentine, composite restorations and porcelain ceramics), the faster it is cut by the particle stream (Black 1950; Gabel 1953; Goldstein et al 1994). Therefore many air abrasion compositions are not suitable for use with soft dental tissue, such as carious dentine. Dental “air polishing”, employing bicarbonate of soda as an abrasive for calculus removal, has shown selective rapid removal of calculus and cementum, compared to sound dentine and prismatic enamel, yet minimally affected the harder aprismatic surface and EDJ enamel (Boyde 1984). The method was deemed acceptable for cleaning enamel of unwanted stain and detritus, yet unacceptable dentine losses were still noted with this technique, prompting a cautionary warning associated with its use on recession exposed root tissues (Boyde 1984). More recently, other materials based on glycine salts have been developed as cleaners of root surfaces, being less abrasive than sodium bicarbonate based materials. Nonetheless bicarbonate of soda and glycine salts are not suited to use in removal of carious dentine. Therefore, there exists a need for materials that are sufficiently soft to preferentially remove carious dentine and to condition the cavity surface prior to bonding with adhesive materials at the same time.

We have now found that by using a polyalkenoate acid as an abrasive (and potential surface peening) agent, in a conventional air abrasion system, benefits are observed in the conditioning of dental tissue.

Accordingly one aspect the present invention provides the use of a polyalkenoate acid in the manufacture of an air abrasive agent for use in conditioning dental tissue.

In another aspect, the present invention provides an air abrasive agent comprising a polyalkenoate acid for conditioning dental tissue.

An air abrasive agent according to this invention is one suited for use in operative dentistry; that is it is to be used in the restoration of teeth using adhesive materials in, for example, the clinical treatment of dental decay, tooth wear, or the modification of tooth shapes for aesthetic purposes. Furthermore, such agents are applicable to any operative technique requiring cavity preparation and/or the use of an adhesive bonding technique to aid retention of a filling material.

In a further aspect the invention provides a method of conditioning dental tissue, said method comprising applying an air abrasive agent comprising a polyalkenoate acid to the dental tissue.

The phrase “conditioning of dental tissue” as used herein refers to one or more of the following processes: the removal of carious dentine, the removal of bacteria, the removal of hard or soft deposits, the removal of cementum, tooth polishing (stain removal), the etching of the surface of the healthy dentine and/or enamel, impregnation of the surface of the healthy dentine and/or enamel. “Dental tissue” includes teeth, including those teeth present in a person's mouth, such that “conditioning of dental tissue” may take place in a person's mouth. Conditioning is not limited to new dental treatments, the dental tissue may be conditioned during, or following removal, addition or repair of an existing restoration.

The management of the water content at the tooth surface, in the hybrid zone between the tooth and the overlying adhesive film and in the overlying adhesive film itself is a major problem with many of the currently available adhesive systems. The use of a polyalkenoate acid according to the invention is particularly advantageous due to the hygroscopic nature of polyalkenoate acids, which results in a reduced risk of hydrolytic breakdown of the adhesive since residual water is effectively removed. In this aspect of the invention the polyalkenoate acid may be applied separately from or together with an air abrasive agent. In this embodiment the air abrasive agent may be a conventional air abrasive agent such as alumina or bioactive glass etc or an air abrasive agent according to the invention. In a particularly preferred embodiment, the polyalkenoate acid is applied in an aqueous solution, preferably dissolved in an aqueous irrigation solution co-localised to the air abrasive gas stream.

Preferred polyalkenoate acids include polyacrylic acid and polymaleic acid.

Although polyacrylic acid has been used as a conditioner and/or an adhesion promoter in adhesive dental materials previously, it has always been used in liquid form. The use of polyalkenoate acids such as polyacrylic acid in the manufacture of an air abrasive agent for use in conditioning dental tissue enables the polyalkenoate acid to be used to treat dental caries by helping to remove carious dentine.

Polyacrylic acid (CAS number: 9003-01-4, chemical formula: (C₃H₄O₂)ₙ) and polymaleic acid (CAS number: 26099-09-2, chemical formula: (C₄H₄0₄)ₙ) are polymeric acids comprising carboxylic acid groups. They are both available in a range of chain lengths and molecular weights. Generally, the longer the chain length the harder the polyalkenoate acid therefore polyalkenoate acids of differing chain lengths may be used for different applications, enabling the hardness of the air abrasive agent to be adjusted. In one embodiment of the present invention polyacrylic acid with a chain length of from 1,200 to 4,000,000, preferably from 10,000 to 100,000, is used.

Polyalkenoate acids suited for use in the present invention will commonly be in a dry form, e.g. powder or granules, preferably powder.

Additionally, the air abrasive agent of the present invention may comprise other known air abrasive agents, for example bioactive glass, glycine salts, baking soda, Dolomite, alumina and other suitable abrasive agents. Cement formation between the poly-acrylic acid (either as air abrasive agent or from irrigation solution) and reactive glasses e.g. bioactive glass air abrasive particles may occur at the tooth interface following impaction in this air abrasion application.

Thus, in another aspect, the invention also provides an air abrasive agent comprising a polyalkenoate acid and at least one of bioactive glass, glycine salts, baking soda, Dolomite (calcium magnesium carbonate CaMg(CO₃)₂), alumina or other suitable abrasive agents.

Typically, alumina particles are used in air abrasion systems. Alumina has a Vickers Hardness of 2300, harder than both tooth enamel and dentine. Thus, when using alumina as the cutting agent the operator must carefully control the extent of cutting so as not to damage the tooth. A material having a Vickers Hardness greater than that of enamel will cut through enamel, a material having a Vickers Hardness intermediate between enamel and healthy dentine will cut through the latter only, and a material having a Vickers Hardness intermediate between healthy dentine and carious dentine will cut through the latter only. Thus, by use of a polyalkenoate acid of an appropriate chain length as an air abrasive agent, and/or by varying the amounts of hardening agents and/or softening agents added to the polyalkenoate acid, the skilled man will be able to prepare air abrasive agents capable of conditioning dental tissue by removal of carious dentine.

TABLE 1

| | Vicker's Hardness Numbers. |
|---|---|
| Demineralised dentine model | not recordable |
| Enamel | 300 |
| Dentine (sound) | 70 |
| Dentine (carious) | 0-55 |

In addition to being able to tune the hardness of the polyalkenoate acid, the size and/or shape of the polyalkenoate acid particles may be adjusted to suit the contours and hardness of the dental tissue to be treated as well. Therefore in one embodiment of the present invention the particles are from 20 microns to 120 microns in diameter. Commonly the polyalkenoate acid particles are irregular in shape.

Preferably, when applied through a conventional air abrasion system the polyalkenoate acid removes the soft carious tissue and remains on the surface of the healthy, hard dental tissue thereby conditioning the dental tissue. Following air abrasion with polyalkenoate acid the conditioned surface of the dental tissue may have a filling material, e.g. a glass ionomer cement, applied directly to it. In this embodiment the use of polyalkenoate acid will permit the filling material to set more quickly and will afford a better bond with the dental tissue. The use of polyalkenoate acid may also permit formation of a better surface shape for the filling material. Alternatively, following air abrasion with polyalkenoate acid the conditioned surface of the dental tissue may have an adhesive applied to it followed by a resin composite.

Therefore in a further aspect of the present invention there is provided a method of treatment and/or prophylaxis of a person suffering from or susceptible to a dental disorder, defined herein to include dental caries, pain, tooth wear, dentine hyper-sensitivity and dental tissue congenital malformations, which method comprises contacting the affected area with an air abrasive agent comprising a polyalkenoate acid. Preferably the method further comprises applying an adhesive, and/or a resin composite, a glass ionomer cement or a combination thereof to the affected area. Following the application of the resin composite, the resin may be cured using electromagnetic radiation.

In a further aspect of the present invention there is provided a method of treatment and/or prophylaxis of a person suffering from dental caries which method comprises, removing the carious dentine from the affected area, optionally etching the affected area with acid, and contacting the affected area with an air abrasive agent comprising a polyalkenoate acid. Preferably the method further comprises applying an adhesive, and/or a resin composite, a glass ionomer cement or a combination thereof to the affected area. In this embodiment the carious dentine is removed by conventional drilling or by an air abrasive agent such as alumina or bioactive glass, preferably the carious dentine is removed by conventional drilling. The adhesive may be a self-etching adhesive. Following the application of the resin composite, the resin may be cured using electromagnetic radiation.

Adhesives suitable for use in the present invention include all current dental adhesives, for example, light cure adhesives, such as Adper Scotchbond MP™ and Optibond FL™, and self-etching light-cure adhesives such as Clearfil's Protect Bond™, Xeno III™, One-Up Bond F Plus™ and Adper Prompt L-Pop™.

It is to be understood that the present invention covers all combinations of suitable and preferred groups described hereinabove.

1-2. (canceled)

3. The composition of claim 23, wherein the polyalkenoate acid is polyacrylic acid or polymaleic acid.

4. (canceled)

5. The composition according to claim 3, wherein the polyalkenoate acid comprises polyacrylic acid having a molecular weight of from 1,200 to 4,000,000.

6. The composition according to claim 23, wherein the air abrasive agent further comprises at least one of a bioactive glass, a glycine salt, baking soda, Dolomite, alumina or other suitable abrasive agent.

7. (canceled)

8. The composition according to claim 23, wherein the particles of the air abrasive agent are from 20 microns to 120 microns in diameter.

9. The composition according to claim 23, wherein the particles of the air abrasive agent are irregular in shape.

10. A method of conditioning dental tissue, said method comprising applying an air abrasive agent comprising a polyalkenoate acid to the dental tissue.

11. A method according to claim 10, wherein the polyalkenoate acid is polyacrylic acid or polymaleic acid.

12. (canceled)

13. A method according to claim 11, wherein the polyalkenoate acid comprises polyacrylic acid having a molecular weight of from 1,200 to 4,000,000.

14. A method according to claim 10, wherein the air abrasive agent further comprises at least one of a bioactive glass, a glycine salt, baking soda, Dolomite, alumina or other suitable abrasive agent.

15. A method according to claim 10, wherein the dental tissue is conditioned during the treatment of dental caries.

16. A method according to claim 10, wherein the particles of the air abrasive agent are from 20 microns to 120 microns in diameter.

17. A method according to claim 10, wherein the particles of the air abrasive agent are irregular in shape.

18-21. (canceled)

22. The composition of claim 23, wherein the air abrasive agent has an average Vickers Hardness of no more than that of tooth enamel, preferably no more than that of healthy dentine.

23. A dental composition comprising (a) an air abrasive agent comprising a polyalkenoate acid and (b) a propellant.

24. A method of treatment and/or prophylaxis of a person suffering from or susceptible to a dental disorder which method comprises contacting the affected area with a composition according to claim 23.

25. A method of treatment and/or prophylaxis of a person suffering from dental caries which method comprises, removing the carious dentine from the affected area, optionally etching the affected area with acid, and contacting the affected area with a composition according to claim 23.

26. The method of claim 10 which further comprises applying an adhesive to the affected area or dental tissue.

27. The method of claim 26 which further comprises applying a resin composite to the affected area or dental tissue.

28-34. (canceled)

35. A method of treatment and/or prophylaxis of a person suffering from or susceptible to a dental disorder which method comprises applying a conventional air abrasive agent and a polyalkenoate acid, such as polyacrylic acid or polymaleic acid, to the affected area, wherein the polyalkenoate acid is applied as aqueous solution co-localised to the air abrasive gas stream.

36-39. (canceled)

40. The composition of claim 23, wherein the propellant is air.
